(19) United States
(12) Patent Application Publication
(10) Pub. No.: US 2016/0029125 A1
(43) Pub. Date: Jan. 28, 2016

(54) SYSTEM AND METHOD FOR ANTICIPATING ACTIVITY USING EARPHONES WITH BIOMETRIC SENSORS

(71) Applicant: JayBird LLC, Salt Lake City, UT (US)

(72) Inventors: JUDD ARMSTRONG, Parrearra (AU); STEPHEN DUDDY, Moama (AU)

(73) Assignee: JAYBIRD LLC, Salt Lake City, UT (US)

(21) Appl. No.: 14/871,953

(22) Filed: Sep. 30, 2015

Related U.S. Application Data
(63) Continuation-in-part of application No. 14/830,549, filed on Aug. 19, 2015.
Application No. 14/830,549 is a continuation-in-part of application No. 14/221,065, filed on Mar. 20, 2014.
Application No. 14/221,065 is a continuation-in-part of application No. 14/140,414, filed on Dec. 24, 2013.
Application No. 14/140,414 is a continuation-in-part of application No. 14/137,942, filed on Dec. 20, 2013.
Application No. 14/137,942 is a continuation-in-part of application No. 14/137,734, filed on Dec. 20, 2013.
Application No. 14/137,734 is a continuation-in-part of application No. 14/062,815, filed on Oct. 24, 2013.

(51) Int. Cl. H04R 5/04; A61B 5/11; A61B 5/0205; A61B 5/024; G06F 3/16; A63B 24/00

(52) U.S. Cl. CPC H04R 5/04; G06F 3/165; A63B 24/0006; A61B 5/0205; A61B 5/02427; A61B 5/1118; H04R 2460/07

(57) ABSTRACT

Systems and methods are disclosed for anticipating a user's activity using earphones with biometric sensors. In one embodiment, the system includes earphones, including: speakers; a processor; a heartrate sensor electrically coupled to the processor; and a motion sensor electrically coupled to the processor. In this embodiment, the system also includes a memory coupled to a processor and having instructions stored that, when executed by the processor: update a stored archive including historical information associated with the user's past activity, where the archive is updated based, in part, on signals generated by the motion sensor and signals generated by the heartrate sensor; and anticipate a future activity of the user based on the updated archive.

### CROSS-REFERENCE TO RELATED APPLICATIONS

This application is a continuation-in-part of and claims the benefit of U.S. patent application Ser. No. 14/830,549 filed Aug. 19, 2015, titled “Earphones with Biometric Sensors,” the contents of which are incorporated herein by reference in their entirety. This application is also a continuation-in-part of and claims the benefit of U.S. patent application Ser. No. 14/221,065 filed Mar. 20, 2014, titled “System and Method for Anticipating Activity,” which is a continuation-in-part of and claims the benefit of U.S. patent application Ser. No. 14/140,414 filed Dec. 24, 2013, titled “System and Method for Providing an Intelligent Goal Recommendation for Activity Level,” which is a continuation-in-part of U.S. patent application Ser. No. 14/137,942, filed Dec. 20, 2013, titled “System and Method for Providing an Interpreted Recovery Score,” which is a continuation-in-part of and claims the benefit of U.S. patent application Ser. No. 14/137,734, filed Dec. 20, 2013, titled “System and Method for Providing a Smart Activity Score,” which is a continuation-in-part of U.S. patent application Ser. No. 14/062,815, filed Oct. 24, 2013, titled “Wristband with Removable Activity Monitoring Device,” the contents all of which are incorporated herein by reference in their entirety.

### TECHNICAL FIELD

The present disclosure relates to earphones with biometric sensors, and more particularly embodiments describe a systems and methods for anticipating user activity using earphones with biometric sensors.

### BRIEF SUMMARY OF THE DISCLOSURE

According to embodiments of the technology disclosed herein, systems and methods are described for anticipating a user's activity using earphones with biometric sensors. In one embodiment, a system for anticipating a user's activity, includes: a pair of earphones, including: speakers; a processor; a heartrate sensor electrically coupled to processor; and a motion sensor electrically coupled to the processor, where the processor is configured to process electronic input signals from the motion sensor and the heartrate sensor. In this embodiment, the system is configured to: update a stored archive comprising historical information associated with the user's past activity, wherein the archive is updated based, in part, on signals generated by the motion sensor and signals generated by the heartrate sensor; and anticipate a future activity of the user based on the updated archive.

In some embodiments, the system presents media content associated with the anticipated future activity to the user. For example, the stored archive may associate a particular media item (e.g., a video, a song, etc.) with a particular activity (e.g., running, walking, cycling, etc.). In a particular implementation, the media content includes songs associated with a playlist, and presenting the media content to the user includes transmitting audio data associated with the songs to the earphones and playing the songs with the earphone speakers using the transmitted audio data.

In some embodiments, the system displays encouragement to the user for the anticipated future activity, where the displayed encouragement is based on the stored archive and the anticipated future activity. In further embodiments, the system provides a notification associated with the user's anticipated future activity to a social network of the user.

In some embodiments, the system displays on a display a set of target goals associated with the anticipated future activity, where each target goal is based on the stored archive. In implementations of these embodiments, the set of target goals include at least one of a target activity type, a target activity intensity, and a target activity duration.

In some embodiments, the archive is updated based, in part, on determining an activity the user engaged in based on signals generated by the motion sensor. In further embodiments, the archive is updated based, in part, on determining a fatigue level of user while engaged in an activity based on signals generated by the heart rate sensor.

In a particular embodiment, the heartrate sensor is an optical heartrate sensor protruding from a side of the earphone proximal to an interior side of a user's ear when the earphone is worn. In implementations of this embodiment, the optical heartrate sensor is configured to measure the user's blood flow and to output an electrical signal representative of this measurement to the earphones processor. In further implementations of this embodiment, the system calculates a heart rate variability value based on signals received from the optical heartrate sensor, and the archive is updated based, in part, on the calculated heart rate variability.

Other features and aspects of the disclosed method and system will become apparent from the following detailed description, taken in conjunction with the accompanying drawings, which illustrate, by way of example, the features in accordance with embodiments of the disclosure. The summary is not intended to limit the scope of the claimed disclosure, which is defined solely by the claims attached hereto.

### BRIEF DESCRIPTION OF THE DRAWINGS

The present disclosure, in accordance with one or more various embodiments, is described in detail with reference to the following Figures. The Figures are provided for purposes of illustration only and merely depict typical or example embodiments of the disclosure.

FIG. 1 illustrates an example communications environment in which embodiments of the disclosed technology may be implemented.

FIG. 2A illustrates a perspective view of exemplary earphones that may be used to implement the technology disclosed herein.

FIG. 2B illustrates an example architecture for circuitry of the earphones of FIG. 2A.

FIG. 3A illustrates a perspective view of a particular embodiment of an earphone, including an optical heartrate sensor, in accordance with the disclosed technology.

FIG. 3B illustrates a side perspective view of placement of the optical heartrate sensor of the earphones of FIG. 3A when they are worn by a user.

FIG. 3C illustrates a frontal perspective view of placement of the optical heartrate sensor of the earphones of FIG. 3A when they are worn by a user.

FIG. 3D illustrates a cross-sectional view of an over-the-ear configuration of dual-fit earphones in accordance with the disclosed technology.

FIG. 3E illustrates a cross-sectional view of an over-the-ear configuration of the dual-fit earphones of FIG. 3D.

FIG. 3F illustrates a cross-sectional view of an under-the-ear configuration of the dual-fit earphones of FIG. 3D.

FIG. 4A is a block diagram illustrating an example computing device that may be used to implement embodiments of the disclosed technology.

FIG. 4B illustrates modules of an example activity monitoring application that may be used to implement embodiments of the disclosed technology.

FIG. 5 is an operational flow diagram illustrating a method of prompting a user to adjust the placement of earphones in the user's ear to ensure accurate biometric data collection by the earphones' biometric sensors.

FIG. 6 illustrates an example system for anticipating user activity.

FIG. 7 illustrates an example apparatus for anticipating user activity.

FIG. 8A is an operational flow diagram illustrating an example method for anticipating user activity.

FIG. 8B illustrates an example embodiment of a stored archive used for anticipating activity, including an archive table.

FIG. 9 is an operational flow diagram illustrating an example method for anticipating user activity, including providing a notification and target goals to the user.

FIG. 10 illustrates an activity display that may be associated with an activity display module of the activity monitoring application of FIG. 4B.

FIG. 11 illustrates a sleep display that may be associated with a sleep display module of the activity monitoring application of FIG. 4B.

FIG. 12 illustrates an activity recommendation and fatigue level display that may be associated with an activity recommendation and fatigue level display module of the activity monitoring application of FIG. 4B.

FIG. 13 illustrates a biological data and intensity recommendation display that may be associated with a biological data and intensity recommendation display module of the activity monitoring application of FIG. 4B.

FIG. 14 illustrates an example computing module that may be used to implement various features of the technology disclosed herein.

### DETAILED DESCRIPTION

Previous generation activity tracking devices generally did not anticipate activity. Currently available activity anticipation devices now add functionality that anticipates activities entered into a calendar. One issue is that currently available activity anticipation devices do not anticipate activity based on past performance. Another issue is that currently available solutions do not provide encouragement, notifications, or target goals for an activity that are specifically tailored specifically to a user's measured performance.

The present disclosure addresses the aforementioned problems and is directed toward systems and methods for anticipating activity. In particular embodiments, the systems and methods are directed to earphones with biometric sensors that are used to anticipate activity.

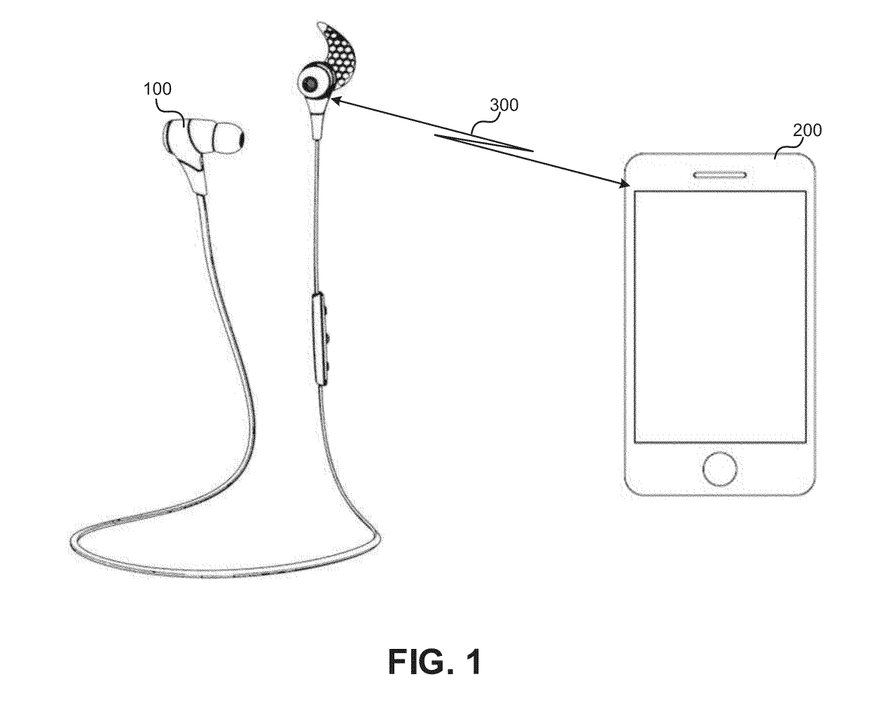

FIG. 1 illustrates an example communications environment in accordance with an embodiment of the technology disclosed herein. In this embodiment, earphones 100 communicate biometric and audio data with computing device 200 over a communication link 300. The biometric data is measured by one or more sensors (e.g., heart rate sensor, accelerometer, gyroscope) of earphones 100. Although a smartphone is illustrated, computing device 200 may comprise any computing device (smartphone, tablet, laptop, smartwatch, desktop, etc.) configured to transmit audio data to earphones 100, receive biometric data from earphones 100 (e.g., heartrate and motion data), and process the biometric data collected by earphones 100. In additional embodiments, computing device 200 itself may collect additional biometric information that is provided for display. For example, if computing device 200 is a smartphone it may use built in accelerometers, gyroscopes, and a GPS to collect additional biometric data.

Computing device 200 additionally includes a graphical user interface (GUI) to perform functions such as accepting user input and displaying processed biometric data to the user. The GUI may be provided by various operating systems known in the art, such as, for example, iOS, Android, Windows Mobile, Windows, Mac OS, Chrome OS, Linux, Unix, a gaming platform OS, etc. The biometric information displayed to the user can include, for example a summary of the user's activities, a summary of the user's fitness levels, activity recommendations for the day, the user's heart rate and heart rate variability (HRV), and other activity related information. User input that can be accepted on the GUI can include inputs for interacting with an activity tracking application further described below.

In preferred embodiments, the communication link 300 is a wireless communication link based on one or more wireless communication protocols such as BLUETOOTH, ZIGBEE, 802.11 protocols, Infrared (IR), Radio Frequency (RF), etc. Alternatively, the communications link 300 may be a wired link (e.g., using any one or a combination of an audio cable, a USB cable, etc.)

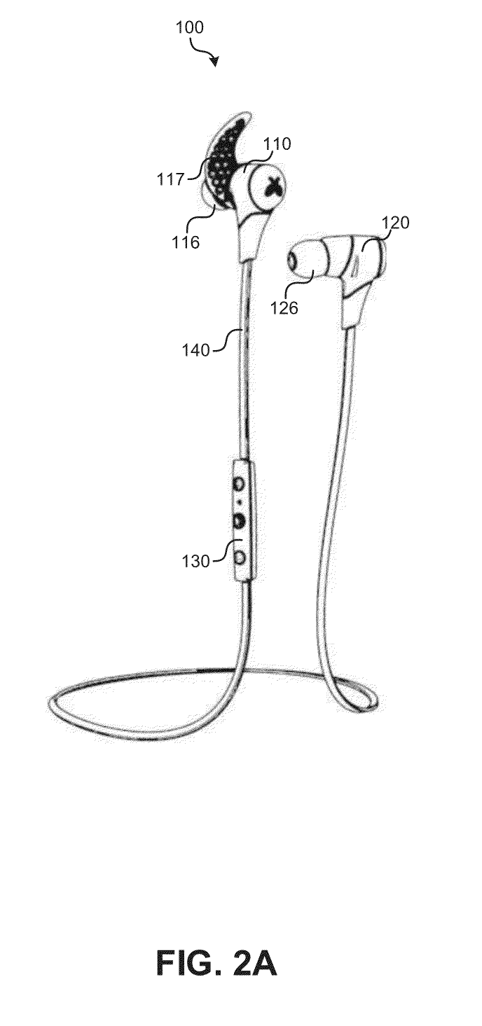

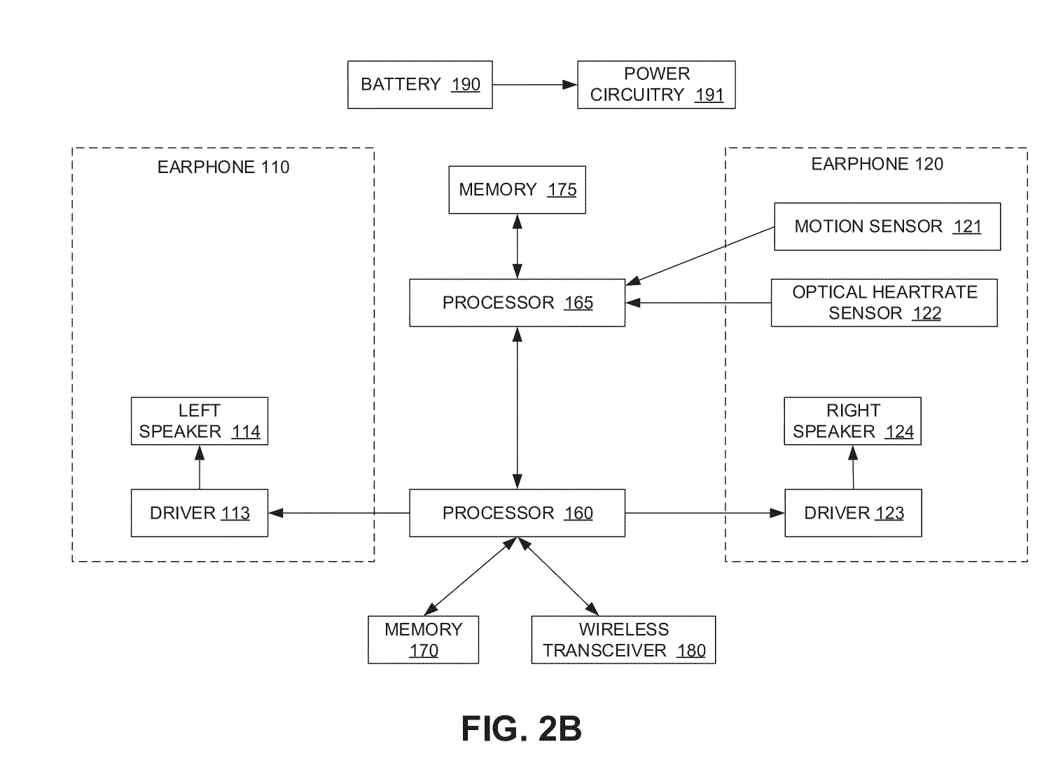

With specific reference now to earphones 100, FIG. 2A is a diagram illustrating a perspective view of exemplary earphones 100. FIG. 2A will be described in conjunction with FIG. 2B, which is a diagram illustrating an example architecture for circuitry of earphones 100. Earphones 100 comprise a left earphone 110 with tip 116, a right earphone 120 with tip 126, a controller 130 and a cable 140. Cable 140 electrically couples the right earphone 110 to the left earphone 120, and both earphones 110-120 to controller 130. Additionally, each earphone may optionally include a fin or ear cushion 117 that contacts folds in the outer ear anatomy to further secure the earphone to the wearer's ear.

In embodiments, earphones 100 may be constructed with different dimensions, including different diameters, widths, and thicknesses, in order to accommodate different human ear sizes and different preferences. In some embodiments of earphones 100, the housing of each earphone 110, 120 is rigid shell that surrounds electronic components. For example, the electronic components may include motion sensor 121, optical heartrate sensor 122, audio-electronic components such as drivers 113, 123 and speakers 114, 124, and other circuitry (e.g., processors 160, 165, and memories 170, 175). The rigid shell may be made with plastic, metal, rubber, or other materials known in the art. The housing may be cubic shaped, prism shaped, tubular shaped, cylindrical shaped, or otherwise shaped to house the electronic components.

The tips 116, 126 may be shaped to be rounded, parabolic, and/or semi-spherical, such that it comfortably and securely fits within a wearer's ear, with the distal end of the tip contacting an outer rim of the wearer's outer ear canal. In some embodiments, the tip may be removable such that it may be exchanged with alternate tips of varying dimensions, colors, or designs to accommodate a wearer's preference and/or fit more closely match the radial profile of the wearer's outer ear canal. The tip may be made with softer materials such as rubber, silicone, fabric, or other materials as would be appreciated by one of ordinary skill in the art.

In embodiments, controller 130 may provide various controls (e.g., buttons and switches) related to audio playback, such as, for example, volume adjustment, track skipping, audio track pausing, and the like. Additionally, controller 130 may include various controls related to biometric data gathering, such as, for example, controls for enabling or disabling heart rate and motion detection. In a particular embodiment, controller 130 may be a three button controller.

The circuitry of earphones 100 includes processors 160 and 165, memories 170 and 175, wireless transceiver 180, circuity for earphone 110 and earphone 120, and a battery 190. In this embodiment, earphone 120 includes a motion sensor 121 (e.g., an accelerometer or gyroscope), an optical heartrate sensor 122, and a right speaker 124 and corresponding driver 123. Earphone 110 includes a left speaker 114 and corresponding driver 113. In additional embodiments, earphone 110 may also include a motion sensor (e.g., an accelerometer or gyroscope), and/or an optical heartrate sensor.

A biometric processor 165 comprises logical circuits dedicated to receiving, processing and storing biometric information collected by the biometric sensors of the earphones. More particularly, as illustrated in FIG. 2B, processor 165 is electrically coupled to motion sensor 121 and optical heartrate sensor 122, and receives and processes electrical signals generated by these sensors. These processed electrical signals represent biometric information such as the earphone wearer's motion and heartrate. Processor 165 may store the processed signals as biometric data in memory 175, which may be subsequently made available to a computing device using wireless transceiver 180. In some embodiments, sufficient memory is provided to store biometric data for transmission to a computing device for further processing.

During operation, optical heartrate sensor 122 uses a photoplethysmogram (PPG) to optically obtain the user's heart rate. In one embodiment, optical heartrate sensor 122 includes a pulse oximeter that detects blood oxygenation level changes as changes in coloration at the surface of a user's skin. More particularly, in this embodiment, the optical heartrate sensor 122 illuminates the skin of the user's ear with a light-emitting diode (LED). The light penetrates through the epidermal layers of the skin to underlying blood vessels. A portion of the light is absorbed and a portion is reflected back. The light reflected back through the skin of the user's ear is then obtained with a receiver (e.g., a photodiode) and used to determine changes in the user's blood oxygen saturation (SpO2) and pulse rate, thereby permitting calculation of the user's heart rate using algorithms known in the art (e.g., using processor 165). In this embodiment, the optical sensor may be positioned on one of the earphones such that it is proximal to the interior side of a user's tragus when the earphones are worn.

In various embodiments, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In further embodiments, logic circuits of processor 165 may further detect, calculate, and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

During audio playback, earphones 100 wirelessly receive audio data using wireless transceiver 180. The audio data is processed by logic circuits of audio processor 160 into electrical signals that are delivered to respective drivers 113 and 123 of left speaker 114 and right speaker 124 of earphones 110 and 120. The electrical signals are then converted to sound using the drivers. Any driver technologies known in the art or later developed may be used. For example, moving coil drivers, electrostatic drivers, electret drivers, orthodynamic drivers, and other transducer technologies may be used to generate playback sound.

The wireless transceiver 180 is configured to communicate biometric and audio data using available wireless communications standards. For example, in some embodiments, the wireless transceiver 180 may be a BLUETOOTH transmitter, a ZIGBEE transmitter, a Wi-Fi transmitter, a GPS transmitter, a cellular transmitter, or some combination thereof. Although FIG. 2B illustrates a single wireless transceiver 180 for both transmitting biometric data and receiving audio data, in an alternative embodiment, a transmitter dedicated to transmitting only biometric data to a computing device may be used. In this alternative embodiment, the transmitter may be a low energy transmitter such as a near field communications (NFC) transmitter or a BLUETOOTH low energy (LE) transmitter. In implementations of this particular embodiment, a separate wireless receiver may be provided for receiving high fidelity audio data from an audio source. In yet additional embodiments, a wired interface (e.g., micro-USB) may be used for communicating data stored in memories 165 and 175.

FIG. 2B also shows that the electrical components of headphones 100 are powered by a battery 190 coupled to power circuity 191. Any suitable battery or power supply technologies known in the art or later developed may be used. For example, a lithium-ion battery, aluminum-ion battery, piezo or vibration energy harvesters, photovoltaic cells, or other like devices can be used. In embodiments, battery 190 may be enclosed in earphone 110 or earphone 120. Alternatively, battery 102 may be enclosed in controller 130. In embodiments, the circuitry may be configured to enter a low-power or inactive mode when earphones 100 are not in use. For example, mechanisms such as, for example, an on/off switch, a BLUETOOTH transmission disabling button, or the like may be provided on controller 130 such that a user may manually control the on/off state of power-consuming components of earphones 100.

It should be noted that in various embodiments, processors 160 and 165, memories 170 and 175, wireless transceiver 180, and battery 190 may be enclosed in and distributed throughout any one or more of earphone 110, earphone 120, and controller 130. For example, in one particular embodiment, processor 165 and memory 175 may be enclosed in earphone 120 along with optical heartrate sensor 122 and motion sensor 121. In this particular embodiment, these four components are electrically coupled to the same printed circuit board (PCB) enclosed in earphone 120. It should also be noted that although audio processor 160 and biometric processor 165 are illustrated in this exemplary embodiment as separate processors, in an alternative embodiment the functions of the two processors may be integrated into a single processor.

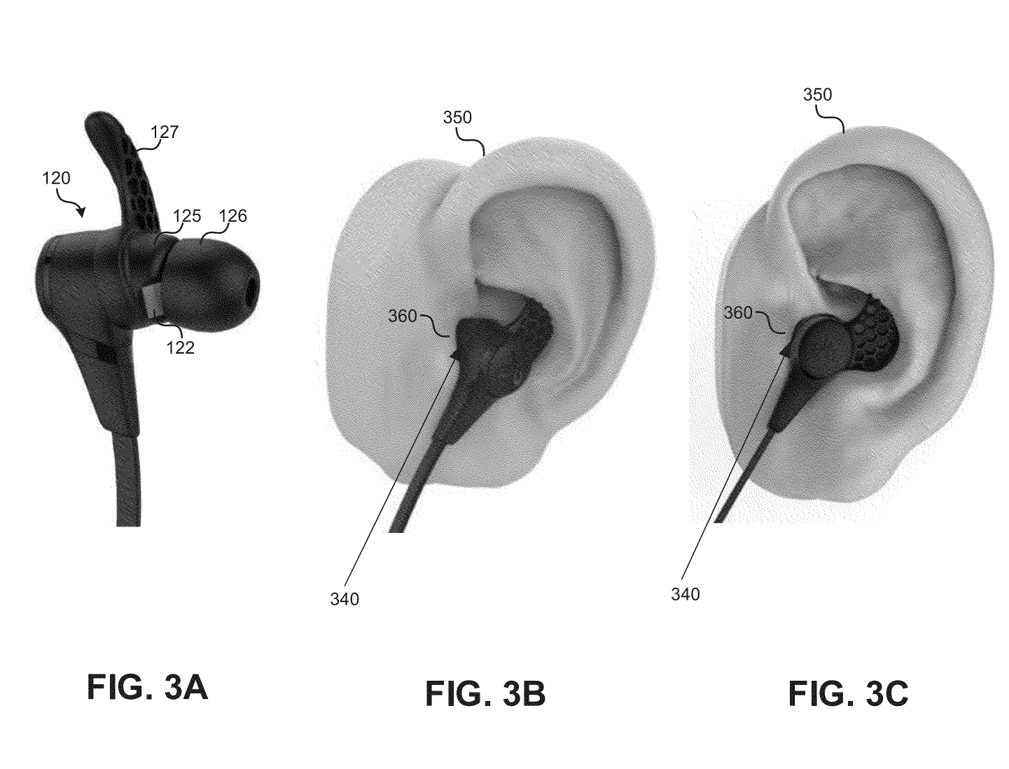

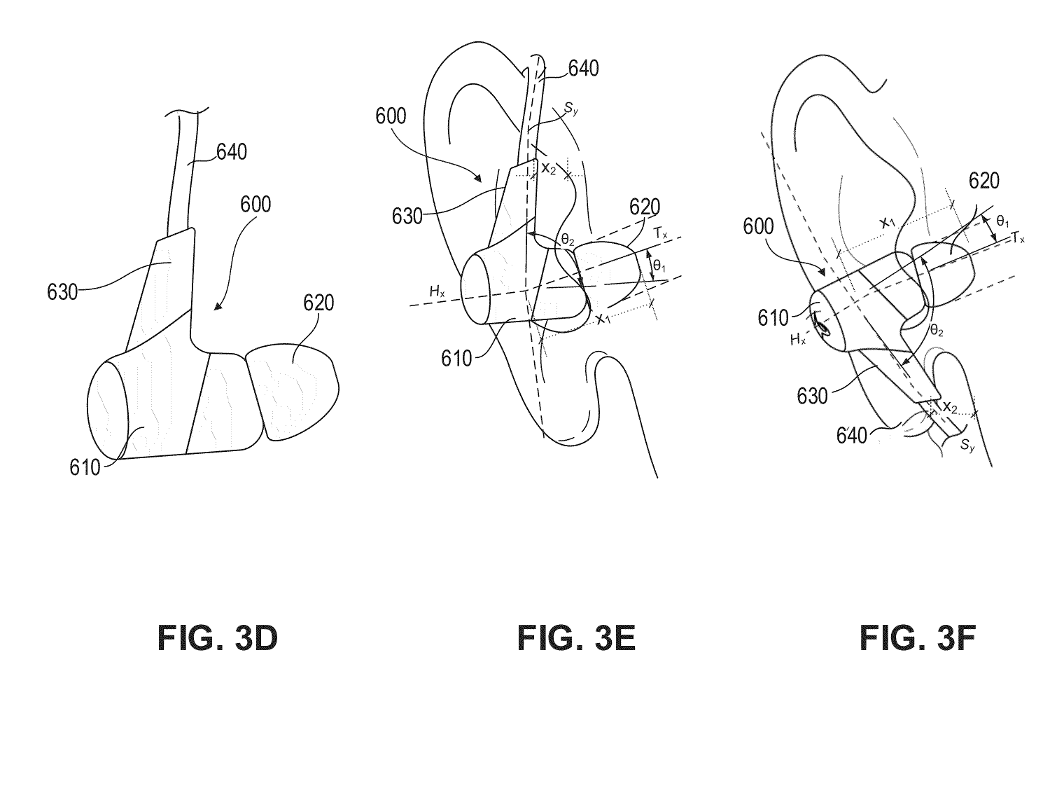

FIG. 3A illustrates a perspective view of one embodiment of an earphone 120, including an optical heartrate sensor 122, in accordance with the technology disclosed herein. FIG. 3A will be described in conjunction with FIGS. 3B-3C, which are perspective views illustrating placement of heartrate sensor 122 when earphone 120 is worn in a user's ear 350. As illustrated, earphone 120 includes a body 125, tip 126, ear cushion 127, and an optical heartrate sensor 122. Optical heartrate sensor 122 protrudes from a frontal side of body 125, proximal to tip 126 and where the earphone's nozzle (not shown) is present. FIGS. 3B-3C illustrate the optical sensor and ear interface 340 when earphone 120 is worn in a user's ear 350. When earphone 120 is worn, optical heartrate sensor 122 is proximal to the interior side of a user's tragus 360.

In this embodiment, optical heartrate sensor 122 illuminates the skin of the interior side of the ear's tragus 360 with a light-emitting diode (LED). The light penetrates through the epidermal layers of the skin to underlying blood vessels. A portion of the light is absorbed and a portion is reflected back. The light reflected back through the skin is then obtained with a receiver (e.g., a photodiode) of optical heartrate sensor 122 and used to determine changes in the user's blood flow, thereby permitting measurement of the user's heart rate and HRV.

In various embodiments, earphones 100 may be dual-fit earphones shaped to comfortably and securely be worn in either an over-the-ear configuration or an under-the-ear configuration. The secure fit provided by such embodiments keeps the optical heartrate sensor 122 in place on the interior side of the ear's tragus 360, thereby ensuring accurate and consistent measurements of a user's heartrate.

FIGS. 3D and 3E are cross-sectional views illustrating one such embodiment of dual-fit earphones 600 being worn in an over-the-ear configuration. FIG. 3F illustrates dual-fit earphones 600 in an under-the-ear configuration.

As illustrated, earphone 600 includes housing 610, tip 620, strain relief 630, and cord or cable 640. The proximal end of tip 620 mechanically couples to the distal end of housing 610. Similarly, the distal end of strain relief 630 mechanically couples to a side (e.g., the top side) of housing 610. Furthermore, the distal end of cord 640 is disposed within and secured by the proximal end of strain relief 630. The longitudinal axis of the housing, Hₓ, forms angle θ₁ with respect to the longitudinal axis of the tip, Tₓ. The longitudinal axis of the strain relief, S_{y}, aligns with the proximal end of strain relief 630 and forms angle θ₂ with respect to the axis Hₓ. In several embodiments, θ₁ is greater than 0 degrees (e.g., Tₓ extends in a non-straight angle from Hₓ, or in other words, the tip 620 is angled with respect to the housing 610). In some embodiments, θ₁ is selected to approximate the ear canal angle of the wearer. For example, θ₁ may range between 5 degrees and 15 degrees. Also in several embodiments, θ₂ is less than 90 degrees (e.g., S_{y} extends in a non-orthogonal angle from Hₓ, or in other words, the strain relief 630 is angled with respect to a perpendicular orientation with housing 610). In some embodiments, θ₂ may be selected to direct the distal end of cord 640 closer to the wearer's ear. For example, θ₂ may range between 75 degrees and 89 degrees.

As illustrated, x₁ represents the distance between the distal end of tip 620 and the intersection of strain relief longitudinal axis S_{y} and housing longitudinal axis Hₓ. One of skill in the art would appreciate that the dimension x₁ may be selected based on several parameters, including the desired fit to a wearer's ear based on the average human ear anatomical dimensions, the types and dimensions of electronic components (e.g., optical sensor, motion sensor, processor, memory, etc.) that must be disposed within the housing and the tip, and the specific placement of the optical sensor. In some examples, x₁ may be at least 18 mm. However, in other examples, x₁ may be smaller or greater based on the parameters discussed above.

Similarly, as illustrated, x₂ represents the distance between the proximal end of strain relief 630 and the surface wearer's ear. In the configuration illustrated, θ₂ may be selected to reduce x₂, as well as to direct the cord 640 towards the wearer's ear, such that cord 640 may rest in the crevice formed where the top of the wearer's ear meets the side of the wearer's head. In some embodiments, θ₂ may range between 75 degrees and 85 degrees. In some examples, strain relief 630 may be made of a flexible material such as rubber, silicone, or soft plastic such that it may be further bent towards the wearer's ear. Similarly, strain relief 630 may comprise a shape memory material such that it may be bent inward and retain the shape. In some examples, strain relief 630 may be shaped to curve inward towards the wearer's ear.

In some embodiments, the proximal end of tip 620 may flexibly couple to the distal end of housing 610, enabling a wearer to adjust θ₁ to most closely accommodate the fit of tip 620 into the wearer's ear canal (e.g., by closely matching the ear canal angle).

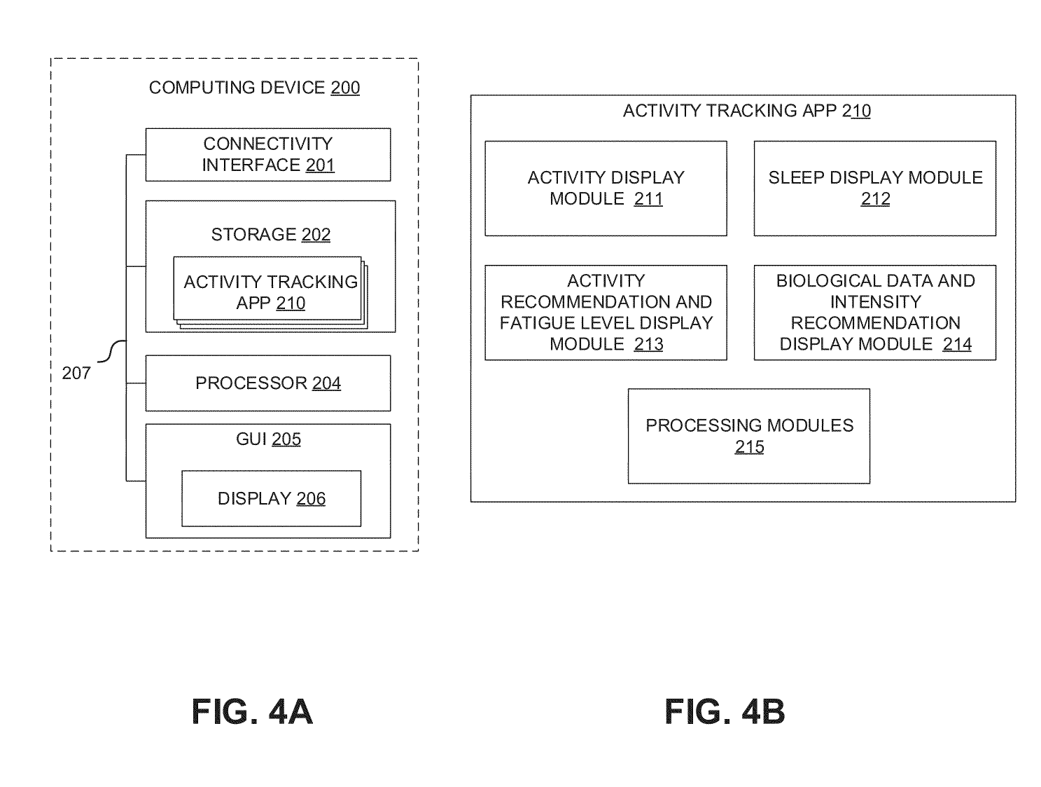

As one having skill in the art would appreciate from the above description, earphones 100 in various embodiments may gather biometric user data that may be used to track a user's activities and activity level. That data may then be made available to a computing device, which may provide a GUI for interacting with the data using a software activity tracking application installed on the computing device. FIG. 4A is a block diagram illustrating example components of one such computing device 200 including an installed activity tracking application 210.

As illustrated in this example, computing device 200 comprises a connectivity interface 201, storage 202 with activity tracking application 210, processor 204, a graphical user interface (GUI) 205 including display 206, and a bus 207 for transferring data between the various components of computing device 200.

Connectivity interface 201 connects computing device 200 to earphones 100 through a communication medium. The medium may comprise a wireless network system such as a BLUETOOTH system, a ZIGBEE system, an Infrared (IR) system, a Radio Frequency (RF) system, a cellular network, a satellite network, a wireless local area network, or the like. The medium may additionally comprise a wired component such as a USB system.

Storage 202 may comprise volatile memory (e.g. RAM), non-volatile memory (e.g. flash storage), or some combination thereof. In various embodiments, storage 202 may store biometric data collected by earphones 100. Additionally, storage 202 stores an activity tracking application 210, that when executed by processor 204, allows a user to interact with the collected biometric information.

In various embodiments, a user may interact with activity tracking application 210 via a GUI 205 including a display 206, such as, for example, a touchscreen display that accepts various hand gestures as inputs. In accordance with various embodiments, activity tracking application 210 may process the biometric information collected by earphones 100 and present it via display 206 of GUI 205. Before describing activity tracking application 210 in further detail, it is worth noting that in some embodiments earphones 100 may filter the collected biometric information prior to transmitting the biometric information to computing device 200. Accordingly, although the embodiments disclosed herein are described with reference to activity tracking application 210 processing the received biometric information, in various implementations various preprocessing operations may be performed by a processor 160, 165 of earphones 100.

In various embodiments, activity tracking application 210 may be initially configured/setup (e.g., after installation on a smartphone) based on a user's self-reported biological information, sleep information, and activity preference information. For example, during setup a user may be prompted via display 206 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by earphones 100 to display activity monitoring information using various modules.

Following setup, activity tracking application 210 may be used by a user to monitor and define how active the user wants to be on a day-to-day basis based on the biometric information (e.g., accelerometer information, optical heart rate sensor information, etc.) collected by earphones 100. As illustrated in FIG. 4B, activity tracking application 210 may comprise various display modules, including an activity display module 211, a sleep display module 212, an activity recommendation and fatigue level display module 213, and a biological data and intensity recommendation display module 214. Additionally, activity tracking application 210 may comprise various processing modules 215 for processing the activity monitoring information (e.g., optical heartrate information, accelerometer information, gyroscope information, etc.) collected by the earphones or the biological information entered by the users. These modules may be implemented separately or in combination. For example, in some embodiments activity processing modules 215 may be directly integrated with one or more of display modules 211-214.

As will be further described below, each of display modules 211-214 may be associated with a unique display provided by activity tracking app 210 via display 206. That is, activity display module 211 may have an associated activity display, sleep display module 212 may have an associated sleep display, activity recommendation and fatigue level display module 213 may have an associated activity recommendation and fatigue level display, and biological data and intensity recommendation display module 214 may have an associated biological data and intensity recommendation display.

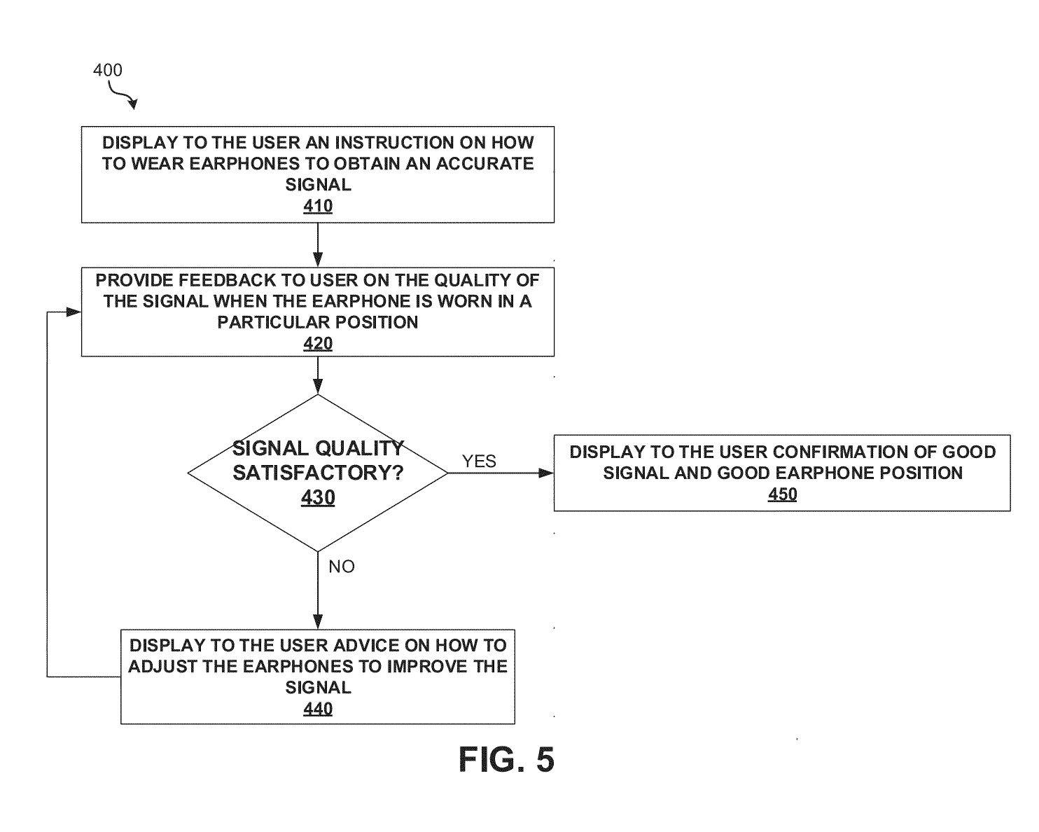

In embodiments, application 210 may be used to display to the user an instruction for wearing and/or adjusting earphones 100 if it is determined that optical heartrate sensor 122 and/or motion sensor 121 are not accurately gathering motion data and heart rate data. FIG. 5 is an operational flow diagram illustrating one such method 400 of an earphone adjustment feedback loop with a user that ensures accurate biometric data collection by earphones 100. At operation 410, execution of application 210 may cause display 206 to display an instruction to the user on how to wear earphones 100 to obtain an accurate and reliable signal from the biometric sensors. In embodiments, operation 410 may occur once after installing application 210, once a day (e.g., when user first wears the earphones 100 for the day), or at any customizable and/or predetermined interval.

At operation 420, feedback is displayed to the user regarding the quality of the signal received from the biometric sensors based on the particular position that earphones 100 are being worn. For example, display 206 may display a signal quality bar or other graphical element. At decision 430, it is determined if the biosensor signal quality is satisfactory for biometric data gathering and use of application 210. In various embodiments, this determination may be based on factors such as, for example, the frequency with which optical heartrate sensor 122 is collecting heart rate data, the variance in the measurements of optical heartrate sensor 122, dropouts in heart rate measurements by sensor 122, the signal-to-noise ratio approximation of optical heartrate sensor 122, the amplitude of the signals generated by the sensors, and the like.

If the signal quality is unsatisfactory, at operation 440, application 210 may cause display 206 to display to the user advice on how to adjust the earphones to improve the signal, and operations 420 and decision 430 may subsequently be repeated. For example, advice on adjusting the strain relief of the earphones may be displayed. Otherwise, if the signal quality is satisfactory, at operation 450, application may cause display 206 to display to the user confirmation of good signal quality and/or good earphone position. Subsequently, application 210 may proceed with normal operation (e.g., display modules 211-214).

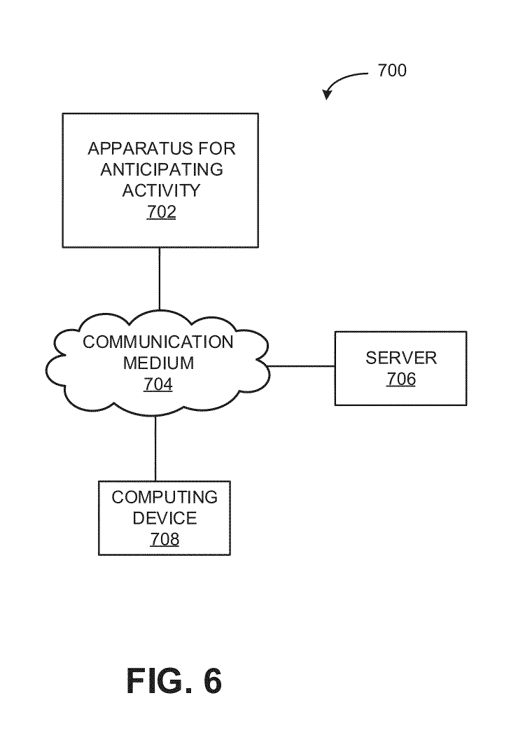

In various embodiments, earphones 100 and computing device 200 may be implemented in a system for anticipating user activity. FIG. 6 is a schematic block diagram illustrating an example system 700 for anticipating activity. System 700 includes an apparatus for anticipating activity 702 (e.g., computing device 200), communication medium 704, server 706, and computing device 708 (e.g., earphones 100).

Communication medium 704 may be implemented in a variety of forms. For example, communication medium 704 may be an Internet connection, such as a local area network (“LAN”), a wide area network (“WAN”), a fiber optic network, internet over power lines, a hard-wired connection (e.g., a bus), and the like, or any other kind of network connection. Communication medium 704 may be implemented using any combination of routers, cables, modems, switches, fiber optics, wires, radio, and the like. Communication medium 704 may be implemented using various wireless standards, such as BLUETOOTH, Wi-Fi, LTE, etc.

Server 706 directs communications made over communication medium 704. Server 706 may be, for example, an Internet server, a router, a desktop or laptop computer, a smartphone, a tablet, a processor, a module, or the like. In one embodiment, server 706 directs communications between communication medium 704 and computing device 708. For example, server 706 may update information stored on computing device 708, or server 706 may send information to computing device 708 in real time.

Computing device 708 may take a variety of forms, such as a desktop or laptop computer, a smartphone, a tablet, a processor, a module, or the like. In addition, computing device 708 may be a module, processor, and/or other electronics embedded in a wearable device such as earphones, a bracelet, a smartwatch, a piece of clothing, and so forth. For example, computing device 708 may be substantially similar to electronics embedded in earphones 100. Computing device 708 may communicate with other devices over communication medium 704 with or without the use of server 706. In one embodiment, computing device 708 includes apparatus 702. In various embodiments, apparatus 702 may be used to perform various processes described herein.

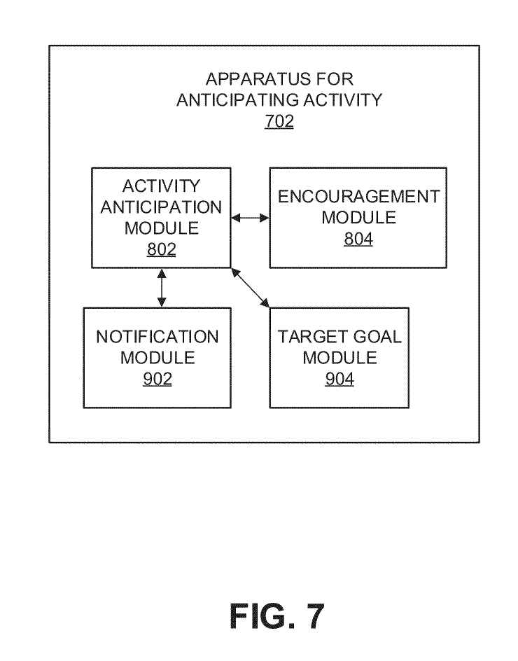

FIG. 7 is a schematic block diagram illustrating an embodiment of an apparatus 702 for anticipating user activity. As illustrated in this particular embodiment, apparatus 702 includes activity anticipation module 802, encouragement module 804, notification module 902, and target goal module 904. Activity anticipation module 802 anticipates an activity based on an archive. The archive includes historical information associated with past user activity. Encouragement module 804 provides encouragement for the activity. The encouragement is based on the archive and the activity. Notification module 902 provides a notification associated with the activity. Target goal module 904 provides a set of target goals for the activity. Activity anticipation module 802, encouragement module 804, notification module 902, and target goal module 904 will be described below in further detail with regard to various processes.

In various embodiments, at least one of activity anticipation module 802, encouragement module 804, notification module 902, and target goal module 904 is embodied in earphones 100. In various embodiments, any of the modules described herein may be embodied in earphones 100 and connect to other modules described herein via communication medium 704.

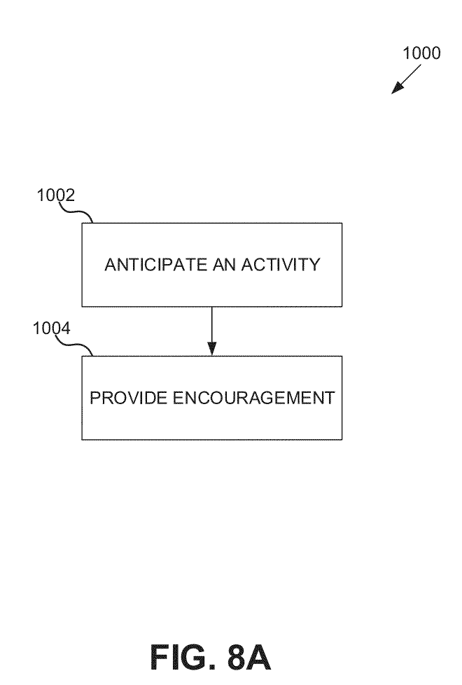

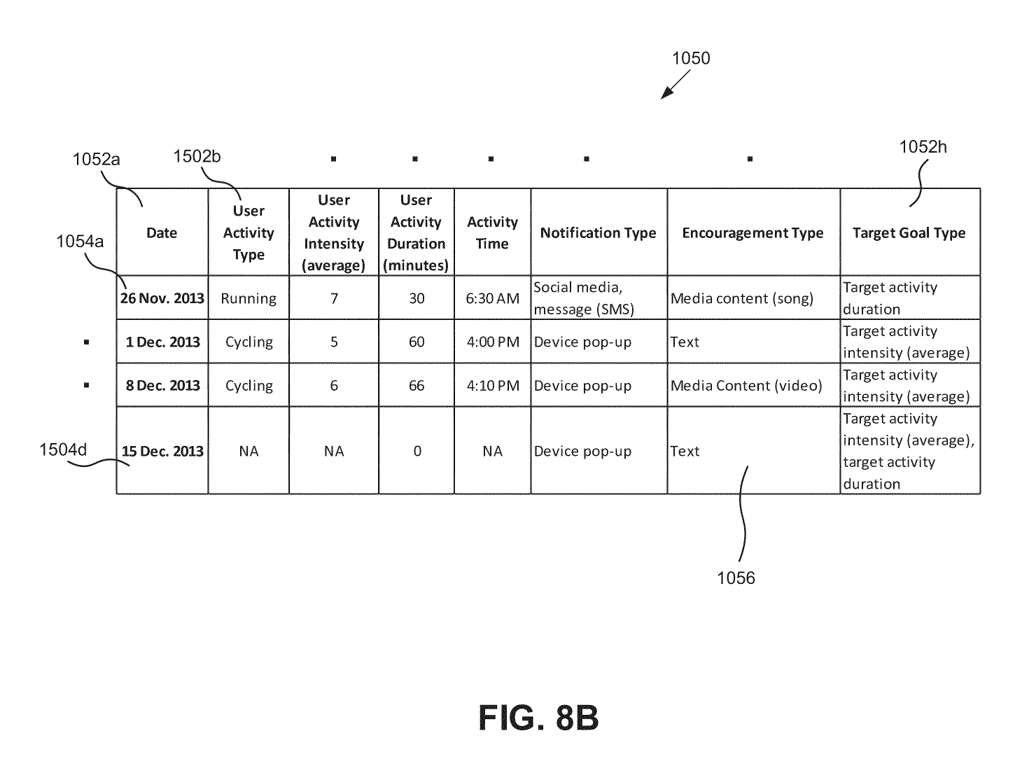

FIG. 8A is an operational flow diagram illustrating example method 1000 for anticipating a user's activity in accordance with an embodiment of the present disclosure. The operations of method 1000 anticipate the user's activity and provide encouragement that is tuned specifically to the user's past performance achievements that are associated with the activity. This aids in providing encouragement that is specifically tailored to the user and that helps the user achieve peak performance in the users' activities. In one embodiment, apparatus 702 and earphones 100 perform various operations of method 1000.

In one embodiment of method 1000, a movement of a user is monitored to identify a user activity type from a set of reference activity types, a user activity intensity from a set of reference activity intensities, and an activity duration for the user activity type or the user activity intensity. For example, the user's movement may be monitored by processing signals generated by motion sensor 121 of earphones 100. Examples of reference activity types include activities such as running, walking, sleeping, swimming, bicycling, skiing, surfing, resting, working, and so on. In embodiments, the user's movement may be further monitored using a global positioning receiver of a mobile device (e.g., a smartphone) such as an Assisted-GPS receiver. The global positioning receiver may be used to gather information associated with the user's location and the user's speed.

In embodiments, the activity duration may be an elapsed time during which the user participated in the user activity type. In addition, the activity duration may be an elapsed time during which the user participated in the user activity at a particular user activity intensity. In various embodiments, the user activity type, user activity intensity, and activity duration are determined using motion sensors (e.g., accelerometer, gyroscope, etc.) and other sensors (e.g., a heart-rate monitor). For example, the user activity type, user activity intensity, and activity duration may be determined by processing signals received from motion sensor 121 and heartrate sensor 122 of earphones 100.

With reference again to FIG. 8A, at operation 1002 of method 1000 a user activity is anticipated based on a stored archive. The archive includes historical information associated with the user's past activity. For example, the archive may include information about the timing of the user's past activity—e.g., time and date information of the activity. In embodiments, the archive includes historical information about the movement that occurred during the past activity. For example, the archive may include historical information about past user activity types, past user activity intensities, and past activity durations. As noted above, this historical information may have been gathered by processing signals generated by motion sensors and other types of sensors (e.g., the sensors of earphones 100, and any additional sensors of device 200) attached to the user during an activity. Additionally, the historical information may have been gathered by the GPS receiver of a mobile device. For example, the historical information may include speed and location data gathered using the GPS receiver.

The archive, in another embodiment, includes historical information about past fatigue levels and past activity locations of the user, as well as information about persons with whom the past activity was performed. Further, the archive may include historical information about the user's mood or general overall feeling, either mental or physical, before, during, or after the past activity. In one embodiment, the archive includes historical information about notifications associated with the past activity, including notification type and notification content. Moreover, the archive may include information and about encouragement, including type and content, associated with the past activity.

In various embodiments, the stored archive is implemented as a table or series of tables, and contains any number of additional information categories, for example, social media events and responses associated with the activity, past and predicted weather conditions, and so on. FIG. 8B illustrates an example embodiment of an archive, including archive table 1050. Archive table 1050 contains archive rows 1054a-d and archive columns 1052a-h. Each archive row 1054 and archive column 1052 combination includes archive data 1056 that log the user's activities. For example, as illustrated in this particular embodiment, the date of the user's activity, the user activity type, the user activity intensity, the user activity duration, the user activity start type, a notification associated with the user activity, encouragement associated with the user activity, and a target goal type of the user activity may all be logged as archive data in table 1050. In other embodiments, Archive table 1050 may contain additional or different archive rows 1054 or archive columns 1052 than those illustrated in FIG. 8B. The archive, in various embodiments, is stored and updated in apparatus 702 (e.g. computing device 200) or computing device 708 (e.g., earphones 100).

Referring again to operation 1002, anticipating the activity based on the archive may be based on any of the information in the archive. For example, the activity may be anticipated based on timing, location, and date information about past activity. To illustrate, the information may indicate that the user consistently goes running at 6:30 AM each Tuesday morning. Upcoming activity may be anticipated based on the assumption that the user will continue—or desires to continue—the status quo. In this example, the status quo would include going running each Tuesday at 6:30 AM. In one embodiment, the anticipated activity is a specific activity—for example, running. In other embodiments, the anticipated is general—for example, exercise, rest, work, and so on.

The activity, in one embodiment, is anticipated even absent a consistent track record of performance. By way of example, the user may have only participated in the activity one time, but such an activity may still be anticipated to recur periodically at various periods. The activity, in another embodiment, is anticipated even though the user never performed the activity. To illustrate, the user may have an activity calendared (e.g., the user is scheduled to go running each Tuesday at 6:30 AM), but the user may fail to go running several Tuesday mornings. In such an embodiment, the activity is anticipated based on the user's calendar, even though the user did not actually go running on Tuesday at 6:30 AM. In a further example, the activity is anticipated based on various inputs—e.g., from the user or from another source.

Referring again to FIG. 8A, at operation 1004 the user is provided encouragement for the activity. The encouragement is based on the archive and the activity. In general, the encouragement may motivate the user to excel in the activity or to take certain actions related to the activity. The encouragement may take various forms and may be provided before, during, or after the activity. In one embodiment, the encouragement is a communication to the user. In various embodiments, the encouragement may be visually displayed to the user (e.g., using application 210 and/or display 206 of computing device 200). For example, the encouragement may be displayed as a message (e.g., text or audio) to the user telling the user to keep up the good work. In another embodiment, the encouragement may be displayed as a ghost comparison. For example, if the activity is running, the encouragement may visually compare the user's current running performance to a past running performance. This may be in the form of providing a graphical comparison of progress through a route, average rate of speed, calories burned, and so on.

In another embodiment, the displayed encouragement is accompanied by media content. In various implementations, the media content may include, for example, a video, photo, or text that is displayed. In a further embodiment, the media content includes one or more songs, or a playlist of songs. The media content, in one embodiment, is selected based on the archive indicating an association between media content and the activity. In embodiments, the association between a particular user activity and media content may be stored in the archive. In one embodiment, upon anticipating the activity (at operation 1002), the media content associated with that activity is provided.

The media content associated with the activity, in one embodiment, is determined to be the user's favorite media content for the activity. For example, the archive may indicate that the user runs faster when listening to a particular song, or the archive may indicate that the user runs for longer when listening to a particular playlist. In addition, the archive may indicate that the user always goes running when a particular video or song is played, but does not always go running when the video or song is not played. In a further embodiment, the user may designate that that particular media content is the user's favorite. In such cases, the provided encouragement includes the user's favorite media content associated with the anticipated activity. This may aid the user in performing the activity at a higher level and may help motivate the user to undertake the activity in the first place.

For example, the user may have a playlist that the user created specifically for running. The playlist may be specifically designated as a running playlist, or the archive may have information indicating that the user frequently listens to the playlist when the user goes running. When the user is anticipated to begin running, the associated playlist may begin playing (e.g., using earphones 100 and computing device 200).

In various embodiments, the media content may be selected based on a record of the user's online browsing history. Such record of browsing history may be related to various mobile applications or Internet applications (e.g., history stored on a computing device 200). For example, the media content may be selected based on the user's history on Facebook®, Pandora®, SoundCloud®, YouTube®, and so on. The media content may be provided via communication medium 704.

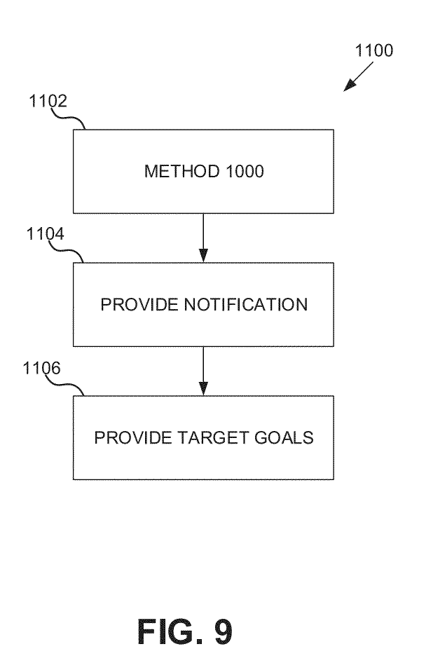

FIG. 9 is an operational flow diagram illustrating example an method 1100 for anticipating activity. In various embodiments, the operations of method 1100 provide a notification associated with the user's activity and provide a set of target goals for the user activity. The notification and the target goals may help motivate the user, and may also help push the user to achieve higher performance. Increased effectiveness may be achieved as both the notification and the target goals are specifically tailored to the user. In one embodiment, apparatus 702 (e.g., computing device 200) and computing device 708 (e.g., earphones 100) perform various operations of method 1100. For example, in various embodiments activity tracking application 210 may be used to provide the notification based on information stored in the archive described above. Method 1100, in various embodiments, includes one or more operations of method 1000, represented at operation 1102.

At operation 1104, a notification associated with the activity is provided to the user. The notification may include information associated with the activity. For example, if there is an anticipated activity type, duration, location, or the like, the notification may indicate such information. In one embodiment, the notification is displayed on computing device 702 (e.g., a mobile device such as a smartphone, television, tablet, smartwatch, or the like). The notification may be in the form of a text message, a pop-up window, an alert, and so forth. The notification, in one embodiment, is provided before the time at which the activity is anticipated to take place. For example, the activity may be anticipated to take place at 6:30 AM, and the notification may be provided the day before at 8:30 PM.

In one embodiment, the notification is provided at a programmable amount of time before the activity. For example, the user may program the notification to be provided two hours before the anticipated activity (e.g., using application 210). In another embodiment, the notification is provided at a predetermined amount of time before the activity based on the activity itself. For example, if the activity is swimming, the user may require sufficient time to get to the location of the pool, change clothes, stretch, etc. This time may be taken into account such that the notification is provided far enough in advance that the user may prepare for the activity and complete the activity during the desired or allotted time. In one embodiment, the notification has a built in snooze function.

The notification, in another embodiment, may be provided via social media. For example, the notification may take the form of a post or status update on Facebook®, a Tweet on Twitter®, or the like. Providing the notification via social media may create accountability for the user in performing the activity. This is because the user may likely have an increased desire to undertake the activity when the user's friends and other connections (or the general public, as the case may be) become aware that the activity is anticipated. Moreover, providing the notification via social media may result in the user receiving encouragement from the user's friends and other connections. For example, upon viewing the notification, the user's social media friends and connections may comment on or otherwise respond to the notification to provide encouragement.

Additionally, providing the notification via social media may allow the user's friends and connections to join the user in the activity, to comment on conditions related to the activity (e.g., weather, road, etc.), or to provide other input. In one embodiment, social media connections who respond to the notification via social media are given the option to directly receive (e.g., via social media, electronic device, etc.) subsequent notifications related to the user's activity. The user may have the ability to select which social media connections are able to receive notifications directly.

In one embodiment, the type of the notification is based on historical information stored in the archive. In such an embodiment, the archive, by way of the historical information may be used to learn the most effective forms of notification for the user. For example, the historical information may indicate that the user more often performs the activity when the notification is posted on the user's Facebook® page. As another example, the historical information may indicate that the user often performs the activity when the notification is delivered to the user's smartphone via text message, but not when the notification is delivered via email. As another example, illustrated by FIG. 8B, the historical information may indicate that the user generally performs the activity when the notification is delivered as a pop-up notification on the user's mobile device.

Similarly, in other embodiments, the historical information may indicate what particular notification content is most effective for the user. For example, the user may respond better to a message calling the user lazy than to a message simply telling the user to undertake the activity. In this manner, the notification may be tailored to the user's preferences and may provide a targeted, effective notification.

Referring again to FIG. 9, at operation 1106 a set of target goals for the anticipated activity are provided to the user (e.g., via a display). The set of target goals, in various embodiments, includes at least one of a target activity type, a target activity intensity, a target activity distance, and a target activity duration. In various embodiments, a user may achieve a target goal by performing the activity type, reaching the target activity intensity, reaching the target activity distance, and/or reaching the target activity duration.

In various embodiments, each of the target goals is based on the stored archive and the anticipated activity. The target goals may include any type of goal associated with the activity and may vary depending on the nature of the activity that is anticipated. For example, the target goal may be that the user participate in the activity with a particular person (e.g., one of the user's friends) or a pet, that the user feel a particular way during or after the activity, or that the user undertake the activity at a particular location (target location). In various embodiments, the target goal may vary as a function of the anticipated activity. For example, the target location for running may be different than the target location for cycling. As the target goals are based on historical information of the stored archive, the target goals may be tailored to the user, and may facilitate pushing the user beyond the user's previous performance.

By way of example, the stored archive may indicate that the user previously exercised for an activity duration of thirty minutes for a particular activity. To facilitate performance improvement and to push the user, the target goal for the next workout may include a target activity duration of thirty-five minutes for the user's anticipated participation in the same activity, thus extending the activity duration to push the user. As an additional example, the archive may indicate that the user completed a S-mile run at an average user activity intensity of 7.0. The target goal may include an increased target activity intensity of 7.5 for a subsequent run, thereby pushing to user to improve.

In one embodiment, the set of target goals includes a combination of a target activity type, target activity intensity, target activity distance, and target activity duration. For example, the set of target goals may include that the user run for forty-five minutes at high intensity. The set of target goals in another embodiment, includes multiple target activity types, with each target activity type having an associated target activity intensity and an associated target activity duration. This may facilitate cross-training.

In various embodiments, the displayed target goals may be based on the user's expected fatigue level (e.g., based on fatigue level previously detected). For example, a higher fatigue level may correspond to a lower target activity intensity or a lower target activity duration, while a lower fatigue level may correspond to a higher target activity intensity or a higher target activity duration. The fatigue level may be detected in various ways. In one example, the fatigue level is detected by calculating a heart rate variability (HRV) of the user using optical heartrate sensor 122 (discussed above in reference to FIG. 2B). When the HRV is more consistent (i.e., steady, consistent amount of time between heartbeats), for example, the fatigue level may be higher. In other words, with a higher fatigue level, the body is typically less fresh and less well-rested. When HRV is more sporadic (i.e., amount of time between heartbeats varies largely), the fatigue level may be lower. In various embodiments, the fatigue level is described in terms of an HRV score.

HRV may be measured in a number of ways (e.g., as discussed above in reference to FIGS. 2B and 3A-3C). Measuring HRV, in one embodiment, involves optical heartrate sensor 122 measuring changes in blood flow. Light reflected back through the skin of the user's ear may be obtained with a receiver (e.g., a photodiode) and used to determine changes in the user's blood flow, thereby permitting calculation of the user's heart rate using algorithms known in the art. Using the data collected by sensor 122, processor 165 may calculate the HRV based on a time domain methods, frequency domain methods, and other methods known in the art that calculate HRV based on data such as the mean heart rate, the change in pulse rate over a time interval, and other data used in the art to estimate HRV. In other embodiments, HRV may be measured using electrocardiography (ECG) or photoplethysmography (PPG) sensors mounted on other parts of the user's body, such as, for example, sensors mounted on the wrist, finger, ankle, leg, arm, or chest.

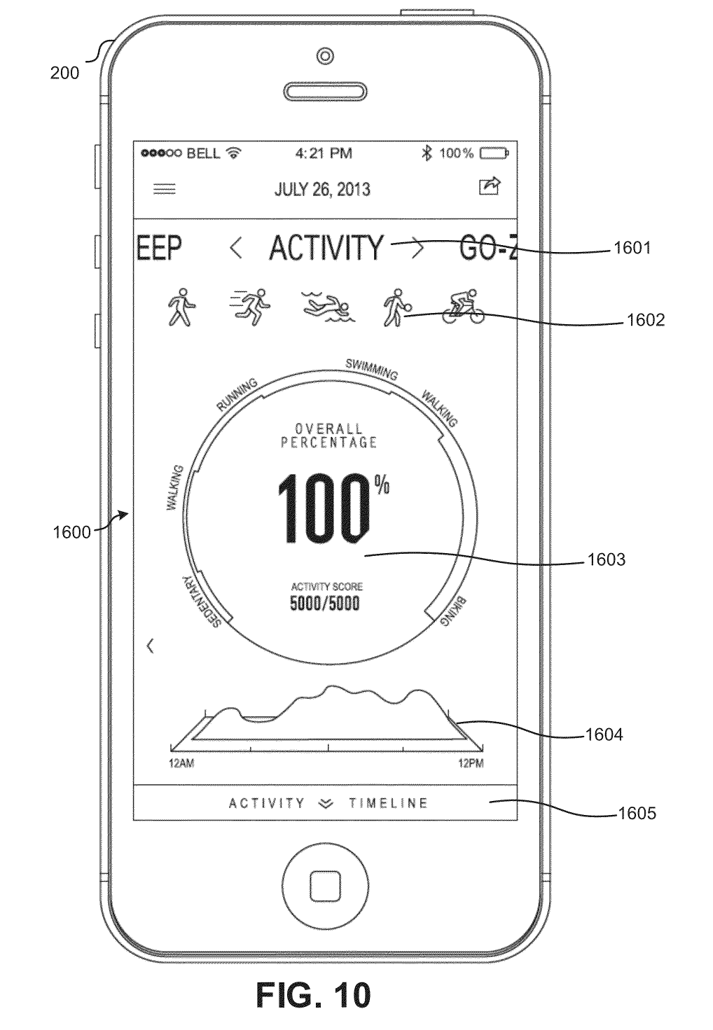

FIGS. 10-13 illustrate a particular implementation of a GUI for activity tracking application 210 comprising displays associated with each of display modules 211-214. In various embodiments, the GUI of activity tracking application 210 may be used to provide encouragements, notifications, and target goal recommendations to the user based on an anticipated activity.

FIG. 10 illustrates an activity display 1600 that may be associated with an activity display module 211. In various embodiments, activity display 1600 may visually present to a user a record of the user's activity. As illustrated, activity display 1600 may comprise a display navigation area 1601, activity icons 1602, activity goal section 1603, live activity chart 1604, and activity timeline 1605. As illustrated in this particular embodiment, display navigation area 1601 allows a user to navigate between the various displays associated with modules 211-214 by selecting “right” and “left” arrows depicted at the top of the display on either side of the display screen title. An identification of the selected display may be displayed at the center of the navigation area 1601. Other selectable displays may displayed on the left and right sides of navigation area 1601. For example, in this embodiment the activity display 1600 includes the identification “ACTIVITY” at the center of the navigation area. If the user wishes to navigate to a sleep display in this embodiment, the user may select the left arrow. In implementations where device 200 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 1602 may be displayed on activity display 1600 based on the user's predicted or self-reported activity. For example, in this particular embodiment activity icons 1602 are displayed for the activities of walking, running, swimming, sport, and biking, indicating that the user has performed these five activities. In one particular embodiment, one or more modules of application 210 may estimate the activity being performed (e.g., sleeping, walking, running, or swimming) by comparing the data collected by a biometric earphone's sensors to pre-loaded or learned activity profiles. For example, accelerometer data, gyroscope data, heartrate data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 1600 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

In various embodiments, an activity goal section 1603 may display various activity metrics such as a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 1603 displays that 100% of the activity goal for the day has been accomplished. Further, activity goal section 1603 displays that activities of walking, running, biking, and no activity (sedentary) were performed during the day. This is also displayed as a numerical activity score 5000/5000. In this embodiment, a breakdown of metrics for each activity (e.g., activity points, calories, and duration) for the day may be displayed by selecting the activity.

A live activity chart 1604 may also display an activity trend of the aforementioned metrics (or other metrics) as a dynamic graph at the bottom of the display. For example, the graph may be used to show when user has been most active during the day (e.g., burning the most calories or otherwise engaged in an activity).

An activity timeline 1605 may be displayed as a collapsed bar at the bottom of display 1600. In various embodiments, when a user selects activity timeline 1605, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

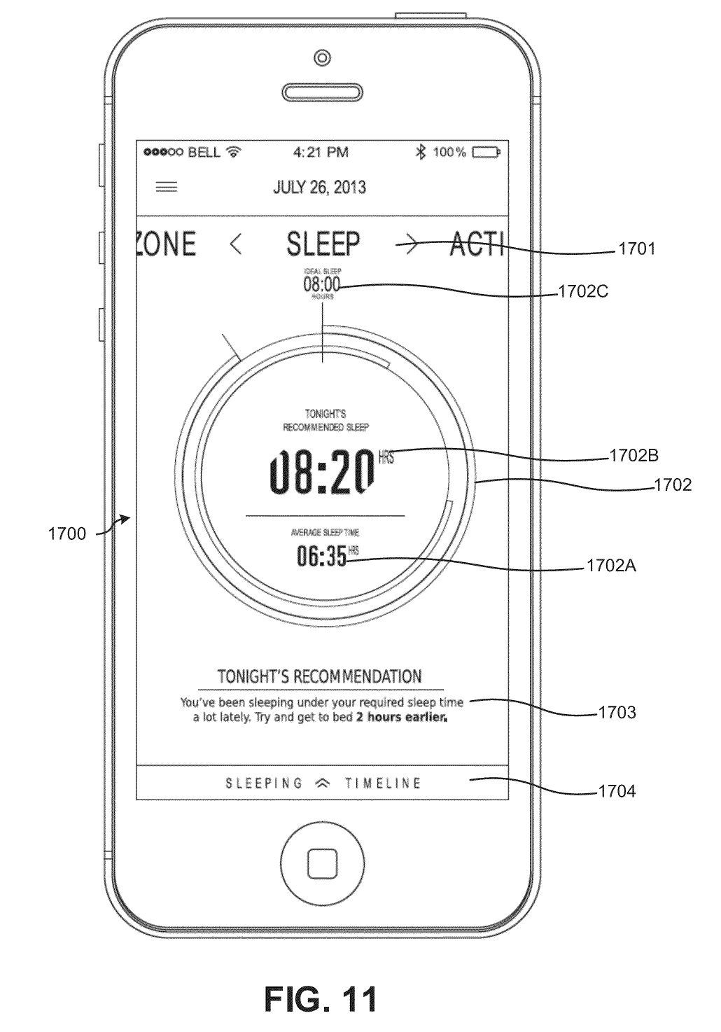

FIG. 11 illustrates a sleep display 1700 that may be associated with a sleep display module 1712. In various embodiments, sleep display 1700 may visually present to a user a record of the user's sleep history and sleep recommendations for the day. It is worth noting that in various embodiments one or more modules of the activity tracking application 1710 may automatically determine or estimate when a user is sleeping (and awake) based on an a pre-loaded or learned activity profile for sleep, in accordance with the activity profiles described above. Alternatively, the user may interact with the sleep display 1700 or other display to indicate that the current activity is sleep, enabling the system to better learn that individualized activity profile associated with sleep. The modules may also use data collected from the earphones, including fatigue level and activity score trends, to calculate a recommended amount of sleep. Systems and methods for implementing this functionality are described in greater detail in U.S. patent application Ser. No. 14/568,835, filed Dec. 12, 2014, and titled “System and Method for Creating a Dynamic Activity Profile”, and U.S. patent application Ser. No. 14/137,942, filed Dec. 20, 2013, titled “System and Method for Providing an Interpreted Recovery Score,” both of which are incorporated herein by reference in their entirety.

As illustrated, sleep display 1700 may comprise a display navigation area 1701, a center sleep display area 1702, a textual sleep recommendation 1703, and a sleeping detail or timeline 1704. Display navigation area 1701 allows a user to navigate between the various displays associated with modules 211-214 as described above. In this embodiment the sleep display 1700 includes the identification “SLEEP” at the center of the navigation area 1701.

Center sleep display area 1702 may display sleep metrics such as the user's recent average level of sleep or sleep trend 1702A, a recommended amount of sleep for the night 1702B, and an ideal average sleep amount 1702C. In various embodiments, these sleep metrics may be displayed in units of time (e.g., hours and minutes) or other suitable units. Accordingly, a user may compare a recommended sleep level for the user (e.g., metric 1702B) against the user's historical sleep level (e.g., metric 1702A). In one embodiment, the sleep metrics 1702A-1702C may be displayed as a pie chart showing the recommended and historical sleep times in different colors. In another embodiment, sleep metrics 1702A-1702C may be displayed as a curvilinear graph showing the recommended and historical sleep times as different colored, concentric lines. This particular embodiment is illustrated in example sleep display 1700, which illustrates an inner concentric line for recommended sleep metric 1702B and an outer concentric line for average sleep metric 1702A. In this example, the lines are concentric about a numerical display of the sleep metrics.

In various embodiments, a textual sleep recommendation 1703 may be displayed at the bottom or other location of display 1700 based on the user's recent sleep history. A sleeping detail or timeline 1704 may also be displayed as a collapsed bar at the bottom of sleep display 1700. In various embodiments, when a user selects sleeping detail 1704, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 1704 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

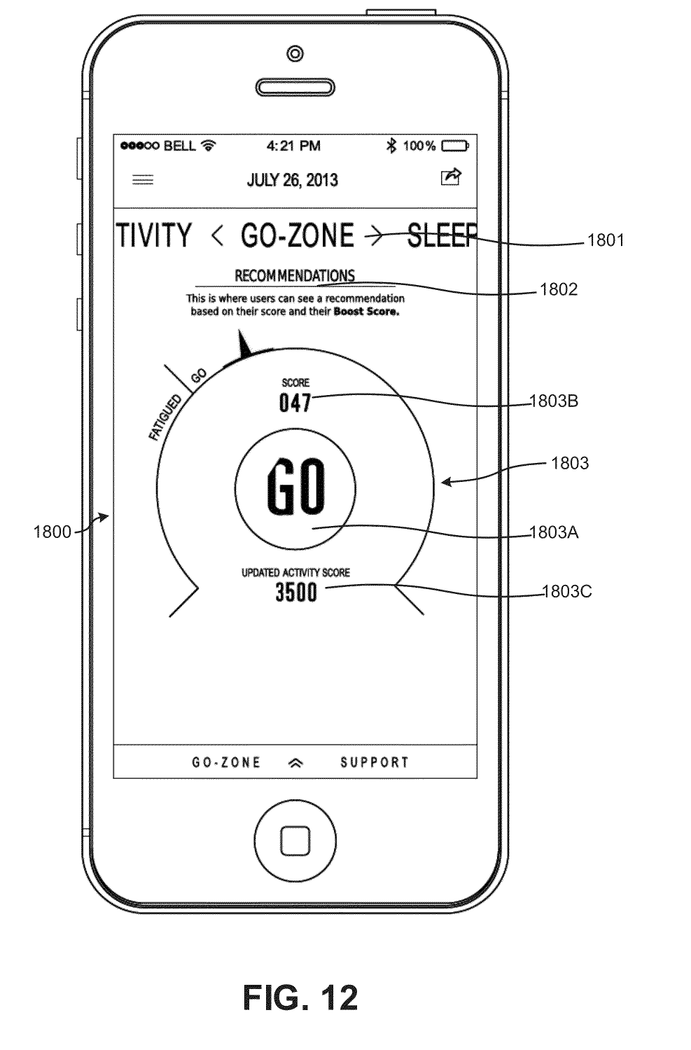

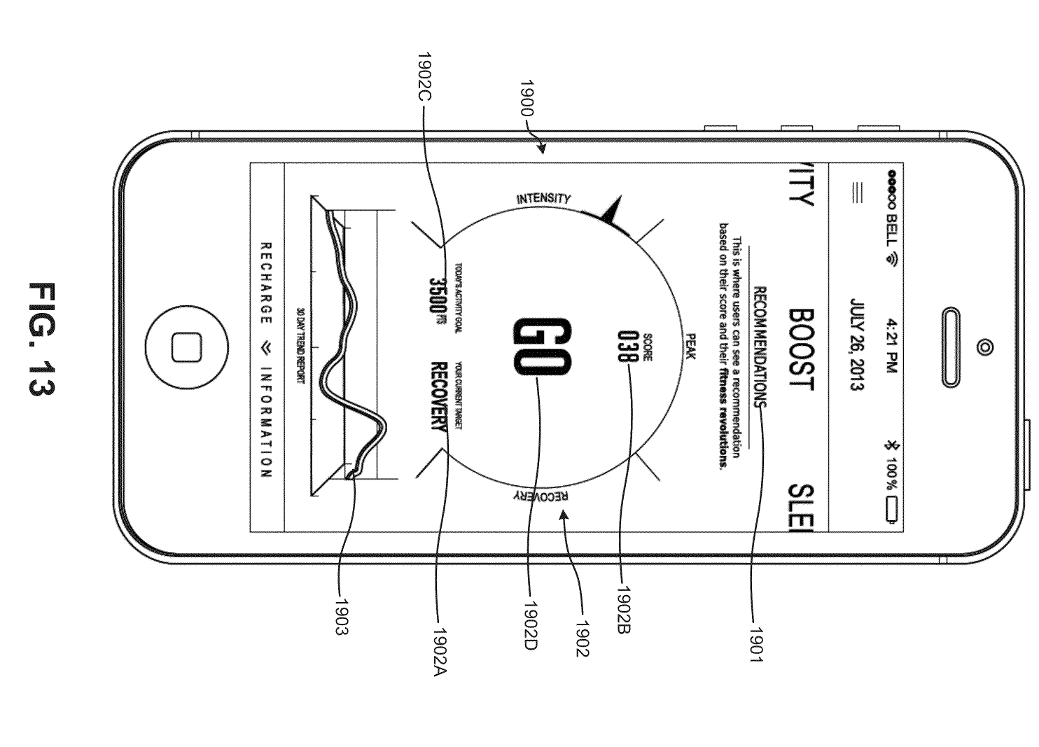

FIG. 12 illustrates an activity recommendation and fatigue level display 1800 that may be associated with an activity recommendation and fatigue level display module 213. In various embodiments, display 1800 may visually present to a user the user's current fatigue level and a recommendation of whether or not engage in activity. It is worth noting that one or more modules of activity tracking application 210 may track fatigue level based on data received from the earphones 100, and make an activity level recommendation. For example, HRV data tracked at regular intervals may be compared with other biometric or biological data to determine how fatigued the user is. Additionally, the HRV data may be compared to pre-loaded or learned fatigue level profiles, as well as a user's specified activity goals. The functionalities of module 213 and display 1800 may be implemented in accordance with embodiments of the systems and methods described herein with reference to FIGS. 7-9.

As illustrated, display 1800 may comprise a display navigation area 1801 (as described above), a textual activity recommendation 1802, and a center fatigue and activity recommendation display 1803. Textual activity recommendation 1002 may, for example, display a recommendation as to whether a user is too fatigued for activity, and thus must rest, or if the user should be active. Center display 1803 may display an indication to a user to be active (or rest) 1803A (e.g., “go”), an overall score 1803B indicating the body's overall readiness for activity, and an activity goal score 1803C indicating an activity goal for the day or other period. In various embodiments, indication 1803A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1800 may be generated by measuring the user's HRV at the beginning of the day (e.g., within 30 minutes of waking up.) For example, the user's HRV may be automatically measured using the optical heartrate sensor 122 after the user wears the earphones in a position that generates a good signal as described in method 400. In embodiments, when the user's HRV is being measured, computing device 200 may display any one of the following: an instruction to remain relaxed while the variability in the user's heart signal (i.e., HRV) is being measured, an amount of time remaining until the HRV has been sufficiently measured, and an indication that the user's HRV is detected. After the user's HRV is measured by earphones 100 for a predetermined amount of time (e.g., two minutes), one or more processing modules of computing device 200 may determine the user's fatigue level for the day and a recommended amount of activity for the day. Activity recommendation and fatigue level display 1800 is generated based on this determination.

In further embodiments, the user's HRV may be automatically measured at predetermined intervals throughout the day using optical heartrate sensor 122. In such embodiments, activity recommendation and fatigue level display 1800 may be updated based on the updated HRV received throughout the day. In this manner, the activity recommendations presented to the user may be adjusted throughout the day.

FIG. 13 illustrates a biological data and intensity recommendation display 1900 that may be associated with a biological data and intensity recommendation display module 214. In various embodiments, display 1900 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1900 may include a textual recommendation 1901, a center display 1902, and a historical plot 1903 indicating the user's transition between various fitness cycles. In various embodiments, textual recommendation 1901 may display a current recommended level of activity or training intensity based on current fatigue levels, current activity levels, user goals, pre-loaded profiles, activity scores, smart activity scores, historical trends, and other bio-metrics of interest. Center display 1902 may display a fitness cycle target 1902A (e.g., intensity, peak, fatigue, or recovery), an overall score 1902B indicating the body's overall readiness for activity, an activity goal score 1902C indicating an activity goal for the day or other period, and an indication to a user to be active (or rest) 1902D (e.g., “go”). The data of center display 1902 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1900 may display a historical plot 1903 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1902B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1903 at the overall score range boundaries. For example, the illustrated historical plot 1903 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

In various embodiments, the various recommendations and measurements of display 1900 may be generated using the methods described above with reference to FIGS. 7-9.

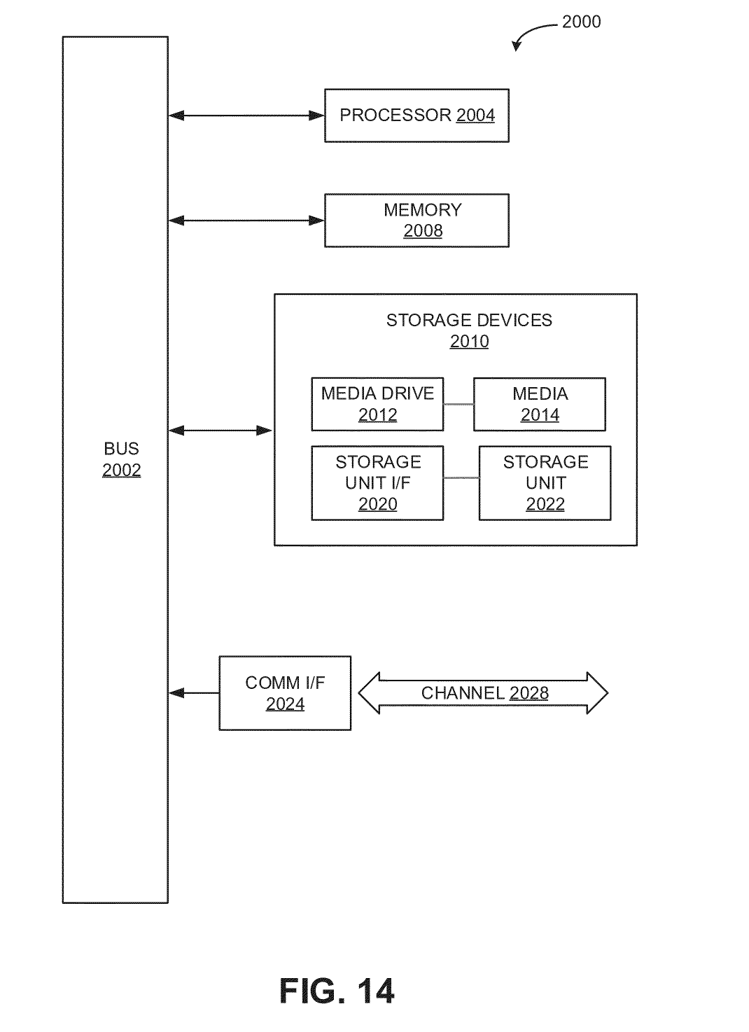

FIG. 14 illustrates an example computing module that may be used to implement various features of the systems and methods for estimating sky probes disclosed herein. As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 14. Various embodiments are described in terms of this example-computing module 2000. After reading this description, it will become apparent to a person skilled in the relevant art how to implement the application using other computing modules or architectures.

Referring now to FIG. 14, computing module 2000 may represent, for example, computing or processing capabilities found within desktop, laptop, notebook, and tablet computers; hand-held computing devices (tablets, PDA's, smart phones, cell phones, palmtops, etc.); mainframes, supercomputers, workstations or servers; or any other type of special-purpose or general-purpose computing devices as may be desirable or appropriate for a given application or environment. Computing module 2000 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 2000 might include, for example, one or more processors, controllers, control modules, or other processing devices, such as a processor 2004. Processor 2004 might be implemented using a general-purpose or special-purpose processing engine such as, for example, a microprocessor, controller, or other control logic. In the illustrated example, processor 2004 is connected to a bus 2002, although any communication medium can be used to facilitate interaction with other components of computing module 2000 or to communicate externally.

Computing module 2000 might also include one or more memory modules, simply referred to herein as main memory 2008. For example, preferably random access memory (RAM) or other dynamic memory, might be used for storing information and instructions to be executed by processor 2004. Main memory 2008 might also be used for storing temporary variables or other intermediate information during execution of instructions to be executed by processor 2004. Computing module 2000 might likewise include a read only memory (“ROM”) or other static storage device coupled to bus 2002 for storing static information and instructions for processor 2004.

The computing module 2000 might also include one or more various forms of information storage mechanism 2010, which might include, for example, a media drive 2012 and a storage unit interface 2020. The media drive 2012 might include a drive or other mechanism to support fixed or removable storage media 2014. For example, a hard disk drive, a solid state drive, a magnetic tape drive, an optical disk drive, a CD, DVD, or Blu-ray drive (R or RW), or other removable or fixed media drive might be provided. Accordingly, storage media 2014 might include, for example, a hard disk, a solid state drive, magnetic tape, cartridge, optical disk, a CD, DVD, Blu-ray or other fixed or removable medium that is read by, written to or accessed by media drive 2012. As these examples illustrate, the storage media 2014 can include a computer usable storage medium having stored therein computer software or data.

In alternative embodiments, information storage mechanism 2010 might include other similar instrumentalities for allowing computer programs or other instructions or data to be loaded into computing module 2000. Such instrumentalities might include, for example, a fixed or removable storage unit 2022 and an interface 2020. Examples of such storage units 2022 and interfaces 2020 can include a program cartridge and cartridge interface, a removable memory (for example, a flash memory or other removable memory module) and memory slot, a PCMCIA slot and card, and other fixed or removable storage units 2022 and interfaces 2020 that allow software and data to be transferred from the storage unit 2022 to computing module 2000.

Computing module 2000 might also include a communications interface 2024. Communications interface 2024 might be used to allow software and data to be transferred between computing module 2000 and external devices. Examples of communications interface 2024 might include a modem or softmodem, a network interface (such as an Ethernet, network interface card, WiMedia, IEEE 802.XX or other interface), a communications port (such as for example, a USB port, IR port, RS232 port BLUETOOTH® interface, or other port), or other communications interface. Software and data transferred via communications interface 2024 might typically be carried on signals, which can be electronic, electromagnetic (which includes optical) or other signals capable of being exchanged by a given communications interface 2024. These signals might be provided to communications interface 2024 via a channel 2028. This channel 2028 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 2008, storage unit 2020, media 2014, and channel 2028. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 2000 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system for anticipating a user's activity, comprising:
a pair of earphones comprising:
speakers;
a processor;
a heartrate sensor electrically coupled to processor; and
a motion sensor electrically coupled to the processor, wherein the processor is configured to process electronic input signals from the motion sensor and the heartrate sensor; and
a non-transitory computer-readable medium operatively coupled to at least one of one or more processors and having instructions stored thereon that, when executed by at least one of the one or more processors, cause the system to:
update a stored archive comprising historical information associated with the user's past activity, wherein the archive is updated based, in part, on signals generated by the motion sensor and signals generated by the heartrate sensor; and
anticipate a future activity of the user based on the updated archive.

2. The system of claim 1, wherein the instructions, when executed by at least one of the one or more processors, further cause the system to present media content associated with the anticipated future activity to the user.

3. The system of claim 2, wherein the media content comprises songs associated with a playlist, and wherein presenting the media content to the user comprises transmitting audio data associated with the songs to the earphones and playing the songs with the earphone speakers using the transmitted audio data.

4. The system of claim 1, wherein the instructions, when executed by at least one of the one or more processors, further cause the system to display on a display a set of target goals associated with the anticipated future activity, wherein each target goal is based on the stored archive, and wherein the set of target goals comprises at least one of a target activity type, a target activity intensity, and a target activity duration.

5. The system of claim 1, wherein the instructions, when executed by at least one of the one or more processors, further cause the system to display on a display encouragement to the user for the anticipated future activity, wherein the displayed encouragement is based on the stored archive and the anticipated future activity.

6. The system of claim 1, further comprising a network interface, wherein the instructions, when executed by at least one of the one or more processors, further cause the system to use the network interface to provide a notification associated with the user's anticipated user activity to a social network of the user.

7. The system of claim 1, wherein the archive is updated based, in part, on determining an activity the user engaged in based on signals generated by the motion sensor.

8. The system of claim 7, wherein the archive is updated based, in part, on determining a fatigue level of user while engaged in an activity based on signals generated by the heart rate sensor.

9. The system of claim 8, wherein the heartrate sensor is an optical heartrate sensor protruding from a side of the earphone proximal to an interior side of a user's ear when the earphone is worn, and wherein the optical heartrate sensor is configured to measure the user's blood flow and to output an electrical signal representative of this measurement to the earphones processor.

10. The system of claim 9, wherein the instructions, when executed by at least one of the one or more processors, further causes the system to calculate a heart rate variability based on signals received from the optical heartrate sensor, and wherein the fatigue level is detected based on the calculated heart rate variability.

11. The system of claim 1, wherein the instructions, when executed by at least one of the one or more processors, further causes the system to determine a location of the user based on a global positioning system, and wherein the anticipated activity of the user is based on the determined location of the user.

12. A method for anticipating a future activity of a user using earphones with biometric sensors, comprising:
monitoring a movement of the user based on electrical signals generated by a motion sensor of the earphones;
detecting a fatigue level of the user based on electrical signals generated by a heart rate sensor of the earphones;
updating a stored archive comprising historical information associated with the user's past activity, wherein the archive is updated based, in part, on the monitored movement and detected fatigue level of the user; and
anticipating a future activity of the user based on the updated archive.

13. The method of claim 12, further comprising: presenting media content associated with the anticipated future activity to the user.

14. The method of claim 13, wherein the media content comprises songs associated with a playlist, and wherein presenting the media content to the user comprises transmitting audio data associated with the songs to the earphones and playing the songs with speakers of the earphones using the transmitted audio data.

15. The method of claim 12, further comprising: displaying on a display encouragement to the user for the anticipated future activity, wherein the displayed encouragement is based on the stored archive and the anticipated future activity.

16. The method of claim 12, further comprising: providing a notification associated with the user's anticipated user activity to a social network of the user.

17. The method of claim 12, further comprising: displaying on a display a set of target goals associated with the anticipated future activity, wherein each target goal is based on the stored archive, and wherein the set of target goals comprises at least one of a target activity type, a target activity intensity, and a target activity duration.

18. The method of claim 12, wherein the motion sensor is an accelerometer.

19. The method of claim 12, wherein the archive is updated based, in part, on determining an activity the user engaged in based on the monitored movement of the user.

20. The method of claim 19, wherein the heartrate sensor is an optical heartrate sensor protruding from a side of the earphone proximal to an interior side of a user's ear when the earphone is worn, and wherein the optical heartrate sensor is configured to measure the user's blood flow and to output an electrical signal representative of this measurement.

21. The method of claim 20, further comprising: calculating a heart rate variability based on signals received from the optical heartrate sensor, and wherein the fatigue level is detected based on the calculated heart rate variability.

22. The method of claim 12, further comprising: determining a location of the user using a global positioning system, and wherein the step of anticipating the activity of the user is based on the determined location of the user.
